(19) United States
(12) Patent Application Publication
(10) Pub. No.: US 2016/0183919 A1
(43) Pub. Date: Jun. 30, 2016

(54) METHOD FOR DISPLAYING STORED HIGH-RESOLUTION DIAGNOSTIC 3-D IMAGE DATA AND 2-D REALTIME SECTIONAL IMAGE DATA SIMULTANEOUSLY, CONTINUOUSLY, AND IN PARALLEL DURING A MEDICAL INTERVENTION OF A PATIENT AND ARRANGEMENT FOR CARRYING OUT SAID METHOD

(71) Applicants: Holger AMTHAUER, Magdeburg (DE); Oliver Stephan GROSSER, Magdeburg (DE)

(72) Inventors: Holger AMTHAUER, Magdeburg (DE); Oliver Stephan GROSSER, Magdeburg (DE)

(21) Appl. No.: 13/806,621

(22) PCT Filed: Jun. 20, 2011
(86) PCT No.: PCT/DE11/01518

§ 371 Date: Mar. 4, 2013

(30) Foreign Application Priority Data
Jun. 22, 2010 (DE) 10 2010 024 851.7

(51) Int. Cl. A61B 8/00; A61B 6/00; A61B 6/03

(52) U.S. Cl. CPC A61B 8/463; A61B 6/032; A61B 6/037; A61B 6/463

(57) ABSTRACT

A method for displaying stored, high-resolution, diagnostic 3D image data and 2D real time image data simultaneously, continuously and in parallel during a medical intervention of a patient utilizes an apparatus including a first unit for storing recorded diagnostic 3D image data sets and for performing dynamic coregistration thereof with a non-diagnostic 3D image data set and of the non-diagnostic 3D image data set with at least one 2D sectional data image set recorded in real time, a second unit for recording the diagnostic 3D image sets, a third unit for substantially instantaneously recording recording the real time 2D sectional image data sets, a fourth unit for displaying the diagnostic 3D image data sets processed by the coregistration, a fifth unit for displaying the real time 2D sectional image data sets, and data transfer connections.

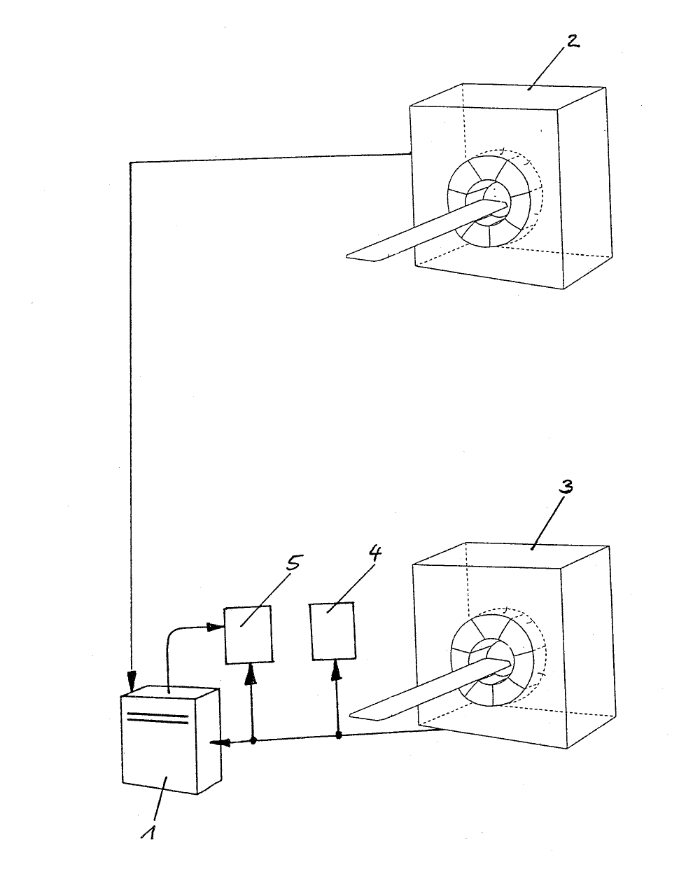

### BACKGROUND OF THE INVENTION

The invention relates to a method for displaying stored, high-resolution, diagnostic 3D image data and 2D real time image data simultaneously, continuously and in parallel during a medical intervention of a patient and to an apparatus for carrying out said method.

DE 10 2006 003 126 A1 describes a method and an apparatus for visualizing three-dimensional objects, particularly in real time. In this method, a three-dimensional image data set of the object is generated, recorded and registered with two-dimensional fluoroscopic images of said object. For visualization purposes, the edges of the object are extracted from the three-dimensional data set and visually overlaid with the edges of the object in the two-dimensional fluoroscopic images. In the so called “overlay method” (common presentation of a 2D image and a projected 3D image) according to DE 10 2006 003 126 A1, the two images are laid one over the other by applying different methods.

The visualization according to 10 2006 003 126 A1 has the disadvantage that after the conducted 2D/3D registration an intuitive presentation and coupling of the two image modalities must be realized in one presentation window (manually by using a joystick operated by the surgeon).

Normally, this step was performed before the procedure according to 10 2006 003 126 A1 in such a way that the pre-operatively obtained 3D image data were presented in parallel to the 2D real time image data on a second visualization system at the moment of intervention (so called “linked cursor” method). In this method, the surgeon/interventional radiologist must manually search in the 3D image data to find the sectional images corresponding to the 2D real time image data. (See, for example, U.S. Pat. No. 6,317,621 B1 and U.S. Pat. No. 6,351,513 B1).

Based on the disclosure in DE 10 2006 003 126 A1, DE 10 2007 051 479 A1 describes a method and an apparatus for displaying the image data of multiple image data sets during a medical intervention, particularly an abdominal one.

The method according to DE 10 2007 051 479 A1 consists of the following steps:
use of at least one three-dimensional image data set of a target region of the intervention recorded before the intervention,
recording of at least one two-dimensional projection data set of the target region,
registration of the three-dimensional image data set of the first step with the two-dimensional image data set of the second step,
synchronous three-dimensional image representation in which at least one set of information of the two-dimensional image data set is transferred into the three-dimensional image data set by means of registration and the angle of vision or the viewing distance of the integrated image representation to the target region can be set differently from the recording angle of viewing or the recording distance of the two-dimensional image data set to the target region.

According to DE 10 2007 051 479 A1 a dual monitor system is used for the synchronous three-dimensional image representation.

The disadvantage of this method is that the preoperatively and intraoperatively recorded 3D/2D image data cannot be coupled in real time.

DE 10 2005 023 167 A1 describes a method and an apparatus for registering 2D projection images of an object relative to a 3D image data set of the same object. In this method, a 3D feature contained in the object and being also identifiable in the 3D image data is symbolically reconstructed from only few 2D projection images. Then, the 3D feature obtained in this way is registered with the 3D image data set by means of 3D-3D registration.

DE 103 59 317 A1 discloses a method for the targeted navigation of a medical instrument, particularly of a catheter, that has been invasively introduced into a cavity organ of the human or animal body, at a pathological location in the cavity organ. In this method, the position of one or several pathological locations is determined by a first image representation of at least a part of the cavity organ obtained in a previous non-invasive examination modality and the image representation is displayed during the subsequent navigation of the instrument together with a continuously angiographically recorded angiography image representation of at least one part of the cavity organ in which the tip of the instrument is located.

This method has the disadvantage that only a continuous angiographically recorded angiography image representation is possible but the preoperatively and intraoperatively recorded sectional images cannot be coupled in real time.

DE 102 10 647 A1 describes a method for displaying images of a medical instrument, particularly a catheter, introduced into an examination region of a patient and said method is performed during a cardiological examination or treatment and consists of the following steps:
using a 3D image data set of the examination region and generating a 3D reconstruction image of this examination region,
recording at least two 2D fluoroscopic images of the examination region that are positioned at a certain angle to each other and show the instrument,
registering the 3D reconstruction image with respect to the 2D fluoroscopic images,
determining the spatial orientation of a catheter tip section and possibly the spatial position of the catheter tip by means of the 2D fluoroscopic images, and
representing the 3D reconstruction image and position-accurate representation of the catheter tip section and possibly of the catheter tip in the 3D reconstruction image on a monitor.

The disadvantage of this method is that preoperatively and intraoperatively recorded image data cannot be coupled in real time.

DE 103 33 543 A1 explains a method for the coupled representation of intraoperative and preoperative images in medical imaging consisting of the following steps:
recording a preoperative image of a region of interest of a patient,
recording an intraoperative image or an image sequence of the region of interest,
registering the preoperative image with the intraoperative image,
coupled representation of the preoperative and intraoperative image,
re-registering the preoperative image, if the preoperative and the intraoperative image do not coincide,
repeating the steps of coupled representation and re-registration until acceptable coincidence is obtained.

For doing this according to DE 103 33 543 A1, from the pre-registered preoperative images a video sequence based on intraoperative images recorded during a periodic physiological motion cycle at defined points in time and/or locations is interpolated between the re-registered images. For this purpose, markers are fixed before recording the pre-operative region so that the registration of the image data is based on markers.

This method has the disadvantage that additional markers must be fixed for the coupled representation of the preoperative and intraoperative image/of the intraoperative image sequence and that the coupling of preoperatively and intraoperatively recorded image data is not possible in real time.

DE 10 2007 051 479 A1 discloses a method and an arrangement for representing image data of several image data sets during a medical, particularly an abdominal, intervention and said method consists of the following steps:

a) using at least one three-dimensional data set of a target region of the intervention recorded before the intervention,
b) recording at least one two-dimensional image data set of the target region,
c) registering the three-dimensional image data set from step a) with the two-dimensional image data set of step b),
d) synchronous three-dimensional image representation in which at least one information of the two-dimensional image data set is transferred by means of the registration into the three-dimensional image data set; the angle of vision or the viewing angle of the integrated image representation to the target region can be set differently from the recording viewing angle or recording distance to the target region. According to DE 10 2007 051 479 A1 a dual monitor system is preferentially used for the synchronous three-dimensional image representation.

The disadvantage of the method described in DE 10 2007 051 479 A1 is that a data or image configuration is used that is not suitable for the CT-guided (or MRT- or US-guided) intervention. 2D projection data sets, which are used for controlling the patient anatomy and the instrument position during the intervention, are generated and registered to the spatial image information (3D data sets). This imaging geometry requires a measuring arrangement according to angiographic systems in which a planar projection recording of the patient is created. In this method, the patient is positioned between the radiator and the radiator detector (e.g. planar detector or image amplifier). In the image-controlled intervention monitored by tomography (CT, MRT, US) this approach cannot be used because projection images (fluoroscopic images according to DE 10 2007 051 479 A1) are not available. The generated data are 2D or 3D sectional image data.

From DE 102 43 162 A1 it is known that in a computer-assisted representation process for a 3D object (9) a 2D basic imaging of the object and a 2D basic representation of a 3D volume data set of the object are determined by a computer and rapidly output as images via an output system. According to DE 102 43 162 A1, the basic imaging and the basic representation are output by the computer simultaneously but separately in terms of location, i.e. to different receiving units.

The disadvantage of this method is that it uses a data/image configuration that is not suited for the CT-guided (or MRT- or US-guided) intervention. The method is based on the application of 2D projection data (fluoroscopic data sets) similar to the application DE 10 2007 051 479 A1. To achieve a better comparability, a 2D projection data set is calculated from a preinterventional 3D sectional image data set. Then, the image information reduced to a 2D data set is represented with the interventional fluoroscopic image for comparing both.

### SUMMARY OF THE INVENTION

The aim of the invention is to develop a method that allows the continuous coupling of preoperatively recorded and stored high-resolution diagnostic 3D image data of the target region of a patient with 2D sectional image data of this target region of the patient, which have been recorded intraoperatively during a medical intervention, in real time during the intervention (navigation according to the “road-map” principle).

Moreover, the task of the invention is to create an arrangement for carrying out said method.

The terms used in describing this invention are defined as follows:

### Diagnostic Image Data:

Diagnostic image data are image data in a quality that is suited for a primary medical diagnosis. Appropriate guidelines for these quality criteria are given by DIN (or international committees), the Association of Statutory Health Insurance Physicians and the German Medical Association. The maintenance of said criteria is the object of a comprehensive quality assurance and the control exercised by audit bodies, e.g. the medical authority specified in the X-Ray Ordinance. The concept of the diagnostic image quality can be considered socially defined for radiation applications on patients.

Non-Diagnostic Image Data (Image Data of Non-Diagnostic Quality):

are all image data that do not fulfill the quality criteria of diagnostic image data. The non-diagnostic image data are generated with the same imaging modalities (e.g. CT, MRT) that are used for creating diagnostic image data. Only the recording reports are adjusted in such a way that they are, for example, created with a considerably reduced measuring time or a significantly lower radiation dose is applied. This adjustment is made to use it for special measuring tasks, here e.g. for employing fluoroscopic image techniques for representing a dynamically changing image information.

### Continuous Generation of the Image Data Set in Real Time:

This means that the non-diagnostic 2D sectional image data (fluoroscopic data) are recorded permanently/continuously. The fluoroscopic 2D sectional image through the examination volume is not frozen image information but each change within the target volume can be seen. This change can be caused either by a physiological process within the sectional plane, e.g. the intestinal motility, or by the instrument pushed forward for interventional purposes.

### Recording an Image Data Set During the Intervention:

The described invention focuses on the area of minimally invasive intervention. In such an intervention an instrument (e.g. a long needle) is introduced via the skin and then pushed forward to a target region (e.g. into a tumor or into a tissue structure that is to be biopsied). The phrase “recording an image data set during the intervention” means that imaging is performed during these interventional activities. As the surgeon (physician) is positioned very close to the patient during imaging and the imaging as a part of the intervention can take several minutes (up to 10 minutes) a considerably reduced dose must be used in case of radiation applications (CT-assisted intervention). Moreover, the imaging performed in parallel shall not considerably prolong the total intervention. For this reason, fluoroscopic imaging is always performed with adapted (accelerated) protocols at the expense of lower diagnostic quality in the image. Diagnostic quality: see diagnostic image data.

### Representation:

The representation is the representation of the results of the coregistration of the non-diagnostic 2D sectional images (fluoroscopic image data) and of the pre-interventional diagnostic 3D image data. Parallel to the fluoroscopic 2D sectional image the spatially corresponding image content of the 3D sectional image data set or of several 3D sectional image data sets of the region to be treated are represented.

### Adaptation of the 3D Sectional Image Data Set to the 2D Sectional Image Data Set:

The spatial registration (identical spatial orientation) of the data to each other is not given a priori; the two examinations (diagnostic and fluoroscopic) are performed at different points in time with deviating positions of the patient, possibly even with different modalities. For this reason, a spatial registration of the image data must be performed. For this purpose, an image information equivalent to the fluoroscopic 2D sectional image data set is searched for in the diagnostic 3D image data set.

### Selection of the Section Plane:

For this selection the image information of the fluoroscopic 2D sectional image data set is compared with the diagnostic 3D data set/data sets until a section through the 3D data is found that shows a minimum deviation.

### Dynamic Coregistration:

The coregistration of the diagnostic 3D data set with the fluoroscopic 2D sectional image data set is carried out permanently. This means that a change of the fluoroscopic 2D sectional images acquired during the intervention and caused, for example, by the changed position of the instrument (needle), results in the representation of the matching section through the diagnostic 3D sectional image data set.

### Continuous Simultaneous Representation:

means that this method permanently compares the fluoroscopic 2D data with the diagnostic 3D data and thus it can always provide an up-to-date diagnostic representation (image information). The comparison must not be triggered and is not performed in discrete, strictly preset time intervals. The updating is only limited by the computer performance.

### Limited Time Window:

The total process of registering diagnostic 3D data on fluoroscopic sectional image data is carried out promptly to the measurement within a time interval of maximally 2 seconds after the measurement of the fluoroscopic sectional image data set. Thus, the registration of the 3D data is almost carried along with a change of the fluoroscopic image information (e.g. by the targeted movement of the patient).

### Short-Term Recording:

This means that the non-diagnostic data set required for the registration can also be obtained during the intervention in a non-fluoroscopic technique (e.g. by rotation angiography). This could also be designated as a kind of one-shot-technique in which a sectional data set (3D or 2D) is recorded singularly. Then, this 3D data set is used for the registration of the image data. In this case said data set, acquired for example by rotation angiography, replaces the fluoroscopic recording in its function.

Data matching of 3D image data, 2D real time image data and 2D real time sectional image data:

The process of registration is controlled by the fluoroscopic 2D real time sectional image data. This data set is continuously and automatically matched with the preinterventionally recorded 3D data sets being provided in diagnostic quality. As a result of this registration one or more section planes the image content of which shows the highest similarity with the fluoroscopic 2D real time sectional image data set is/are selected from the diagnostic data set.

This/These selected section plane/s is/are displayed as a navigation support with diagnostic quality for the interventionist.

### BRIEF DESCRIPTION OF THE DRAWING

The invention is explained in more detail by reference to the drawing, which is a schematic representation of an apparatus of the invention for carrying out the method of the invention.

### DETAILED DESCRIPTION OF THE INVENTION

The apparatus shown in the drawing consists of a unit (1) for storing recorded diagnostic 3D image data sets and for performing the dynamic coregistration, a unit (2) for recording diagnostic 3D image data sets, a unit (3) for the short-term recording of non-diagnostic 3D image data sets and for recording 2D real time sectional image data, a unit (4) for displaying the diagnostic 3D image data sets processed by coregistration, and a unit (5) for displaying the 2D real time sectional data, and data transfer connections are provided between the units (1) and (2), (1) and (3) and between (1), (4) and (5).

The unit (3) of the apparatus of the invention is a magnet resonance tomograph (MRT), computer tomograph (CT) or ultrasonic (US) device for the short-term recording of non-diagnostic 3D image data sets in combination with a fluoroscopic apparatus for recording 2D real time sectional image data.

According to the invention, the unit (4) is a dual-monitor system and the unit (1) is a computer.

The arrangement shown in the drawing is used for displaying stored high-resolution diagnostic 3D image data and 2D real time image data simultaneously, continuously and in parallel during a medical intervention of a patient.

This inventive method is characterized by the following steps:
using at least one high-resolution diagnostic 3D image data set of a target region of the patient recorded before the intervention,
continuously recording at least one 2D sectional image data set of the target region in real time,
capturing at least one non-diagnostic 3D image data set of the target region without diagnostic quality recorded during the intervention,
adapting the representation of the high-resolution diagnostic 3D image data set to the representation of 2D sectional image data set and selecting section planes of the high-resolution diagnostic 3D image data set that correspond to the section planes of the 2D sectional image data set by dynamically coregistering the diagnostic 3D image data set with the non-diagnostic 3D image data set and the non-diagnostic 3D image data set with the 2D sectional image data set, and
continuously and simultaneously displaying the dynamically coregistered diagnostic 3D image data set and the 2D sectional image data set in real time on two devices located next to each other for presenting the 3D image data set and the 2D real time sectional image data in parallel.

According to the invention, the non-diagnostic 3D image data set or the 2D sectional image data set is recorded by computer tomography (CT), magnet resonance tomography (MRT), C-arm X-ray or positron emission tomography (PET) in a limited time window from 0.1 to 0.5 second.

The 2D sectional image data set is fluoroscopically recorded in real time by computer tomography (CT) or magnet resonance tomography (MRT) or is recorded in real time by ultrasound.

According to the invention, the information of the diagnostic 3D image data set, of the non-diagnostic 3D image data set and of the 2D sectional image data set are coregistered mathematically and automatically by a computer.

This invention has the advantage of the continuous coupling of pre-operatively recorded and stored high-resolution diagnostic 3D image data of the target region of a patient with 2D sectional image data of this target region of the patient recorded intraoperatively during a medical intervention in real time during the intervention (navigation according to the “roadmap” principle).

Apart from the pure intervention control in the sense of a roadmap, the inventive apparatus and the inventive method also allow to superinpose additional information, e.g. the pre-planning of the position of radiation catheters to allow an optimized radiation plan for a later brachytherapy. The information superimposed is used for guiding the operator/interventional radiologist during the introduction of the catheter.

Due to a better localization (quasi a navigation support of the surgeon) the inventive apparatus and the inventive method lead to a significant increase of the precision of micro-therapeutic interventions and to minimized risks during such interventions.

All elements presented in the description, the subsequent claims and in the drawing can be essential for the invention both as single elements and in any combination.

1. Method for displaying stored, high-resolution diagnostic 3D image data and 2D real time image data simultaneously, continuously and in parallel during a medical intervention of a patient comprising:
before the intervention recording at least one high-resolution, diagnostic 3D image data set of a target region of a patient,
continuously recording at least one 2D sectional image data set of the target region in real time during the intervention,
recording at least one non-diagnostic 3D image data set of the target region without diagnostic quality during the intervention,
adapting representation of the high-resolution diagnostic 3D image data set to representation of the 2D sectional image data set and selecting section planes of the high-resolution diagnostic 3D image data set that correspond to the section planes of the 2D sectional image data set by dynamically coregistering the diagnostic 3D image data set with the non-diagnostic 3D image data set and the non-diagnostic 3D image data set with the 2D sectional image data set, and
continuously and simultaneously displaying the dynamically coregistered diagnostic 3D image data set and the 2D sectional image data set in real time on two devices located next to each other for presenting the 3D image data and the 2D real time sectional image data in parallel.

2. Method according to claim 1, wherein the non-diagnostic 3D image data set or the 2D sectional image data set is recorded by computer tomography (CT), magnet resonance tomography (MRT), C-arm X-ray or positron emission tomography (PET) in a limited time window ranging from 0.1 to 0.5 second.

3. Method according to claim 1, wherein the 2D sectional image data set is recorded in real time by computer tomography (CT) fluoroscopy magnet resonance tomography (MRT) fluoroscopy or ultrasound.

4. Method according to claim 1, wherein the dynamic coregistering is effected mathematically and automatically by a computer.

5. Apparatus for displaying stored, high-resolution diagnostic 3D image data and 2D real time sectional image data simultaneously, continuously and in parallel during a medical intervention of a patient comprising
a first unit for storing recorded diagnostic 3D image data sets and for performing the dynamic coregistration,
a second unit for recording diagnostic 3D image data sets,
a third unit for substantially instantaneously recording of non-diagnostic 3D image data sets and for recording 2D real time sectional image data,
a fourth unit for displaying the diagnostic 3D image data sets processed by the coregistration, and
a fifth unit for displaying the 2D real time sectional data, and
data transfer connections between the first and second units, between the first and third units, and between the first, fourth and fifth units.

6. Apparatus according to claim 5, wherein the third unit is a magnet resonance tomography or ultrasonic device for the short-term recording of non-diagnostic 3D image data sets in combination with a fluoroscope for recording 2D real time sectional image data.

7. Apparatus according to claim 5, wherein the fourth unit is a dual-monitor system.

8. Apparatus according to claim 5, wherein the first unit is a computer.

9. Apparatus according to claim 5, wherein duration of the substantially instantaneous recording effected by the third unit is 0.1 to 0.5 second.
